(19) United States
(12) Patent Application Publication
(10) Pub. No.: US 2016/0157720 A1
(43) Pub. Date: Jun. 9, 2016

(54) HYBRID FLUORESCENCE-MAGENTIC IMAGING SYSTEM

(71) Applicant: ASPECT IMAGING LTD., SHOHAM (IL)

(72) Inventors: Uri RAPOPORT, Moshav Ben Shemen (IL); Aryeh BATT, Har Hevron (IL)

(73) Assignee: ASPECT IMAGING LTD., SHOHAM (IL)

(21) Appl. No.: 15/041,076

(22) Filed: Feb. 11, 2016

Related U.S. Application Data
(63) Continuation-in-part of application No. 13/943,346, filed on Jul. 16, 2013, now abandoned.

(30) Foreign Application Priority Data
Aug. 15, 2012 (IL) 221490

(51) Int. Cl. A61B 5/00; A61B 5/055

(52) U.S. Cl. CPC A61B 5/0035; A61B 5/055; A61B 5/0071; A61B 5/0077; A61B 5/7264

(57) ABSTRACT

The present invention provides a hybrid imaging system for imaging a volume of interest of a subject, said hybrid system is characterized by:

a. a MRI device;

b. a photon transmitter, introducible within the body of an animal;

c. at least one imaging photon detector located either within or outside said animal, for detecting fluorescence excited within said animal by said transmitted photons; and

d. an image processor adapted to superimpose said MRI image and said at least one photon detector image, generating a rendered MRI image of said volume of interest of the subject.

The hybrid system is configured to substantially simultaneously acquire MRI image and in vivo fluorescence image.

### CROSS-REFERENCE TO RELATED APPLICATIONS

This application is a continuation-in-part of U.S. patent application No. US 13/943,346, filed Jul. 16, 2013. The previous application is incorporated in its entirety by reference.

### FIELD OF THE INVENTION

The present invention generally pertains to a hybrid imaging system and method for an MRI-based system for generating a fused image of an interior portion of an animal by superimposing simultaneously acquired MRI image and fluorescence image, where the fluorescence is excited by light emitted by a photon emitter located within an animal body.

### BACKGROUND OF THE INVENTION

In vivo fluorescence imaging uses a sensitive camera to detect fluorescence emission from fluorophores in whole-body living small animals. To overcome the photon attenuation in living tissue, fluorophores with long emission at the near-infrared (NIR) region are generally preferred, including widely used small indocarbocyanine dyes.

Molecules that absorb in the near infrared (NIR) region, 700-1,000 nm, can be efficiently used to visualize and investigate in vivo molecular targets because most tissues generate little NIR fluorescence. The most common organic NIR fluorophores are polymethines Among them, pentamethine and heptamethine cyanines comprising benzoxazole, benzothaizole, indolyl, 2-quinoline or 4-quinoline have been found to be the most useful.

Fluorescence images enable determination of cells types, cell activity and protein activity; See review by Lowry et al., Molecular Fluorescence, Phosphorescence, and Chemiluminescence Spectrometry Analytical Chemistry, Vol. 80, No. 12, June 15, 4551-4574, 2008, which is incorporated herein as a reference.

MRI or NMR provides images of the locations of particular atomic species in a volume of interest, especially protons. MRI provides good contrast between the different soft tissues of the body, which makes it especially useful in imaging the brain, muscles, the heart, and cancers compared with other medical imaging techniques such as computed tomography (CT) or X-rays. MRI contrast agents may be injected intravenously to enhance the appearance of blood vessels, tumors or inflammation. Contrast agents may also be directly injected into a joint in the case of arthrograms, MRI images of joints.

Patent application US 2010/0113902 discloses an efficient, effective, MRI compatible small bore MRI noninvasive photoplefhysmographic sensor for animals such as small rodents, namely rats and mice. The photoplethysmographic sensor for animals comprising: a non-magnetic sensor coupling attachable to an animal; fiber optic cable coupled to the sensor coupling and configured to deliver a signal to and receive a signal from the animal tissue adjacent the sensor coupling; an opto-electical converter coupled to the fiber optic cable, the converter including a receiver coupled the fiber optic cable portion configured to receive a signal from the animal tissue and including an emitter coupled to the fiber optic portion configured to deliver a signal to the animal tissue; an electronic coupling extending from the opto-electric converter and configured to be coupled to the emitter and the receiver, wherein the electronic coupling is configured to extend outside of the MRI chamber; and a processor coupled to the electronic coupling. However, the energy inducing the fluorescence within the animal is supplied from a source attached to the outside of the animal.

Patent application US 2005/0028482 discloses systems and methods for multi-modal imaging with light and a second form of imaging. Light imaging involves the capture of low intensity light from a light-emitting object. A camera obtains a two-dimensional spatial distribution of the light emitted from the surface of the subject. Software operated by a computer in communication with the camera may then convert two-dimensional spatial distribution data from one or more images into a three-dimensional spatial representation. The second imaging mode may include any imaging technique that compliments light imaging. Examples include magnetic resonance imaging (MRI) and computer topography (CT). An object handling system moves the object to be imaged between the light imaging system and the second imaging system, and is configured to interface with each system. However, the energy inducing the fluorescence within the animal is supplied from a source external to the animal.

Multimodality hybrid imaging system have a number of advantages over sequential imaging. The most important advantage is the ability to simultaneously perform imaging with each modality under identical physiological and geometric conditions.

There have been significant effort in developing hybrid fluorescence-magnetic resonance imaging system. Problems still exist in integrating different imaging modalities, obtaining accurate image co-registration and interpretation of combined imaging data.

Over the last decade, fiber-optic fluorescence imaging has become increasingly versatile as fiber-based devices have declined in size but gained in functionality. It is therefore a long felt need to provide a multi-modality system combining simultaneous MRI and endoscopic fluorescence imaging for preclinical and clinical investigations of tumors where fluorescence is induced from a source within the body of a subject.

### SUMMARY OF THE INVENTION

It is thus an object of the present invention to disclose a hybrid imaging system (100) for imaging a volume of interest of a subject, said hybrid imaging system comprises: a) a magnetic resonance imaging (MRI) device (130) for accommodating said volume of interest of said subject and for acquiring at least one MRI image thereof; b) a photon transmitter (150) selected from the group consisting of an optical fiber, a cannula, a light pipe, a light tube, and any combination thereof; said photon transmitter (150) having one end connected to a light source and a second end introducible into said volume of interest of said subject while said predetermined portion of said subject is placed within said MRI device, said photon transmitter being specifically adapted to being guided from within said predetermined portion of said subject; c) at least one imaging photon detector (140) selected from the group consisting of a CCD array, a camera, a photoconductive detector array, a photovoltaic detector array, a quantum dot array, a superconducting single-photon detector array, a photovoltaic cell array, a phototube array, and any combination thereof; said photon detector (140) located functionally solely outside and substantially proximate to said predetermined portion of said animal body for acquiring at least one fluorescence image of said volume of interest of said subject substantially simultaneously with acquiring of said MRI image, by detecting fluorescence emission from an external surface of said subject, said fluorescence is excited within said subject upon illumination by light transmitted by said photon transmitter; and d) a processor adapted to fuse said acquired MRI image and said at least one acquired fluorescence image, thereby generating a rendered MRI image of said volume of interest of said subject. Wherein said hybrid imaging system substantially simultaneously acquires said at least one magnetic resonance image and said at least one fluorescence images of said volume of interest of said subject.

It is another object of the present invention to disclose the hybrid imaging system as defined in any of the above, wherein said photon transmitter is an optical fiber selected from the group consisting of silica glass fiber, fluorozirconate glass fiber, fluoroaluminate glass fiber, chalcogenide glass fiber, sapphire fiber, and polymer optical fiber.

It is another object of the present invention to disclose the hybrid imaging system as defined in any of the above, wherein said processor utilizes Boolean logic techniques including operators selected from the group consisting of OR, AND, NOT, EXCLUSIVE OR, and any combination of said operators, for facilitating the correlation and fusion of images.

It is another object of the present invention to disclose the hybrid imaging system as defined in any of the above, wherein said processor utilizes fusing techniques including fuzzy logic, the intensity-hue-saturation (IHS) algorithm, the retina-inspired model (RIM) fusion technique and any combination thereof, for facilitating the correlation and fusion of images.

It is another object of the present invention to disclose the hybrid imaging system as defined in any of the above, wherein said photon transmitter is adapted to enter said subject through an orifice selected from the group consisting of a cannula inserted in said subject, a trocar inserted in said subject, a laparoscopy system inserted in said subject, the nose, the mouth, the anus, the vagina, the urethra, the ear, and any combination thereof.

It is another object of the present invention to disclose the hybrid imaging system as defined in any of the above, wherein photons transmitted by said photon transmitter are in a range selected from the group consisting of X-rays, far ultraviolet, near ultraviolet, visible light, near infrared and far infrared.

It is another object of the present invention to disclose the hybrid imaging system as defined in any of the above, wherein magnets in said MRI imaging device are selected from the group consisting of permanent magnets, superconducting magnets, and any combination thereof.

It is also an object of the present invention to disclose a method of imaging a volume of interest of a subject, comprising steps of: a) providing a magnetic resonance imaging (MRI) system (130) for accommodating said volume of interest of said subject and for acquiring at least one MRI image; b) transmitting photons utilizing means selected from the group consisting of an optical fiber, a cannula, a light pipe, a light tube, and any combination thereof; said photon transmitting means utilizes one end thereof connected to a light source and a second end thereof introducible into said volume of interest of said subject while said subject is placed within said MRI device, said photon transmitting means specifically adapted to being guided from within said portion of said animal body; c) detecting at least one photon-induced image utilizing means selected from the group consisting of a CCD array, a camera, a photoconductive detector array, a photovoltaic detector array, a quantum dot array, a superconducting single-photon detector array, a photovoltaic cell array, a phototube array, and any combination thereof; said detecting of at least one photon-induced image being functionally accomplished solely outside and substantially proximate to said volume of interest of said subject for obtaining at least one fluorescence image of said volume of interest of said subject substantially simultaneously with the said acquiring of said MRI image, said detecting of fluorescence emission occurring from an external surface of said subject, said fluorescence being excited within said animal body upon illumination by light transmitted by said photon transmitter; and d) processing and fusing both said at least one MRI image and said at least one acquired fluorescence image, thereby generating a rendered MRI image of said volume of interest of said subject. Wherein said at least one magnetic resonance image and said at least one fluorescence images of said volume of interest of said subject are acquired substantially simultaneously.

It is also an object of the present invention to disclose the method as mentioned in any of the above, additionally comprising a step of selecting the optical fiber from the group consisting of silica glass fiber, fluorozirconate glass fiber, fluoroaluminate glass fiber, chalcogenide glass fiber, sapphire fiber, and polymer optical fiber.

It is also an object of the present invention to disclose the method as mentioned in any of the above, additionally comprising a step of emplacing said fiber within said body through an orifice selected from the group consisting of a cannula inserted in the animal, a trocar inserted in the animal, a laparoscopy system inserted in the animal, the nose, the mouth, the anus, the vagina, the urethra, the ear, and any combination thereof.

It is also an object of the present invention to disclose the method as mentioned in any of the above, additionally comprising a step of selecting the range of said photons from at least one of the group consisting of X-rays, far ultraviolet, near ultraviolet, visible light, near infrared and far infrared.

It is also an object of the present invention to disclose the method as mentioned in any of the above, further comprising a step of selecting Boolean logic operators selected from the group consisting of OR, AND, NOT, EXCLUSIVE OR, and any combination of said operators, for facilitating the correlation and fusion of images.

It is also an object of the present invention to disclose the method as mentioned in any of the above, further comprising a step of selecting fusing techniques including fuzzy logic, the intensity-hue-saturation (IHS) algorithm, the retina-inspired model (RIM) fusion technique and any combination thereof, for facilitating the correlation and fusion of images.

It is also an object of the present invention to disclose the method as mentioned in any of the above, additionally comprising a step of selecting magnets in said MRI imaging device from the group consisting of permanent magnets, superconducting magnets, and any combination thereof.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 schematically illustrates the hybrid system of the present invention according to a preferred embodiment;

FIG. 2 depicts a block diagram of a method of using the hybrid system of the present invention according to a preferred embodiment.

### DETAILED DESCRIPTION OF THE PREFERRED EMBODIEMNTS

The following description is provided, alongside all chapters of the present invention, so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide an MRI-based system and method for generating a rendered image of at least a portion of a living subject.

The terms “modality” refers herein in a non-limiting manner to an attribute of the device of the invention which is that the device is provided with more than one means for generating an image or images. In preferred embodiments, the device is provided with NMR means or modalities to generate images of a subject, such as MRI, and also, the very same device is provided with optical means or modalities for generating images of the same subject. Both NMR means and optical means may generate time resolved images.

The term “volume of interest” refers to a volume within the subject of which an image is desired. The volume of interest thus may be, for example, the entire subject, an organ within the subject, or a specific volume within an organ within the subject (e.g. the site at which a tumor is suspected to exist).

The term “subject” refers to any object or living creature inserted in whole or in part into the static magnetic field of a magnetic resonance imaging (MRI) system in order to obtain at least one magnetic resonance image thereof or therefrom.

The term “detector” refers to an apparatus adapted for measuring the intensity of a signal impinging upon it and transmitting that intensity to a recording device. The detector will in general include all of the necessary electronics to convert the received signal to a current, voltage, or number proportional to the intensity of the signal and means for passing the current, voltage, or number to an appropriate recording device.

The term “fluorescence imaging” refers to fiber optic in vivo imaging, including endoscopic and microendoscopic fiber fluorescence imaging. The components of the fiber optic imaging modality, such as optical fiber and photon detector are nonmagnetic.

The term “probe” is interchangeable with “imaging agent” or “imaging biomarker”, refers to any chemicals, biochemical or biomolecules designed to allow clinicians to improve the imaging of specific organs, tissues, diseases and physiological functions.

The term “fuse” is interchangeable with overlay or superimpose, refers to the act of placing or laying image obtained from one modality over another to obtain a combined image.

The term “substantially” applies hereinafter to a measure being ±25% of the defined value.

In vivo fluorescence imaging uses a sensitive camera to detect fluorescence emission from fluorophores in whole-body living small animals. To overcome the photon attenuation in living tissue, fluorophores with long emission at the near-infrared (NIR) region, 700-1000 nm, are generally preferred because they can be efficiently used to visualize and investigate in vivo molecular targets, since most tissues generate little NIR fluorescence.

One widely used class of fluorophores is small indocarbocyanine dyes. In addition, fluorescent organic, inorganic and biological nanoparticles are used. Another class of probes for in vivo fluorescence imaging is semiconductor nanocrystals or quantum dots. QDs that emit at several different wavelengths can be excited with a single wavelength, and thus are suitable for multiplex detection of multiple targets in a single experiment.

According to some preferred embodiments, dual-modality probes are used, which could be imaged by both MRI and fluorescence imaging modalities. Examples include but are not limited to dendrimer-based nanoprobes, rare-earth nanocrystals, gadolinium-doped zinc oxide quantum dots, Dye-Doped silica nanoparticles et al.

A low-cost, Magnetic Resonance Imaging (MRI) compatible fibre-optic sensor for integration with catheters allowing the detection of contact forces between blood vessel walls and the catheter tip. Three plastic optical-fibres are aligned inside a plastic catheter in a circular pattern. A reflector is attached to a separate small part of the catheter tip, which is connected with a small deformable material to the aligned optical fiber. In this manner a force at the catheter tip leads to a deformation of the elastic material and thus a modulation of the light yields, this is sent and received through the optical fiber. An electronic circuit amplifies the retrieved light signal and the output voltage is used to classify the forces on the tip. The materials used are of the shelf and have a low magnetic susceptibility making this sensor fully MRI-compatible and inexpensive. Preliminary, experimental results demonstrated good force linearity in static loading and unloading conditions. The sensor was also tested in an artificial blood artery showing good dynamic response.

Magnetic resonance imaging (MRI) uses a strong static magnetic field to align nuclear magnetic moment with the field. A varying field, usually with frequencies in the range of 50-200 MHz, is used to systematically alter the alignment of this magnetization. This causes the nuclei to produce a rotating magnetic field detectable by the scanner, and this information is recorded to construct an image of the scanned area of the body. Magnetic field gradients cause nuclei at different locations to rotate at different speeds. By using gradients in different directions, 2D images or 3D volumes can be obtained in any arbitrary orientation. MRI provides good contrast between the different soft tissues of the body, which makes it especially useful in imaging the brain, muscles, the heart, and cancers compared with other medical imaging techniques such as computed tomography (CT) or X-rays. Unlike CT scans or traditional X-rays, MRI does not use ionizing radiation.

Fluorescence images of the tumors enable determination of cells types (senescent, aggressive, etc.) and protein activity, while NMR/MRI enables determination of the location of, especially, protons. Differences between bound and free water, for example, allow determination of edema.

MRI provides good contrast between the different soft tissues of the body, which makes it especially useful in imaging the brain, muscles, the heart, and cancers compared with other medical imaging techniques such as computed tomography (CT) or X-rays. MRI contrast agents may be injected intravenously to enhance the appearance of blood vessels, tumors or inflammation. Contrast agents may also be directly injected into a joint in the case of arthrograms, MRI images of joints.

Combining fluorescence imaging with MRI can help to advance imaging for cancer by overlaying functional information about tumor growth onto an anatomical map provided by MRI.

The system of the present invention provides a system of simultaneously acquiring MRI images and fluorescence images in living subjects of organs, tumors, blood vessels, nerves, or any other objects in the living subject that can be made to fluoresce.

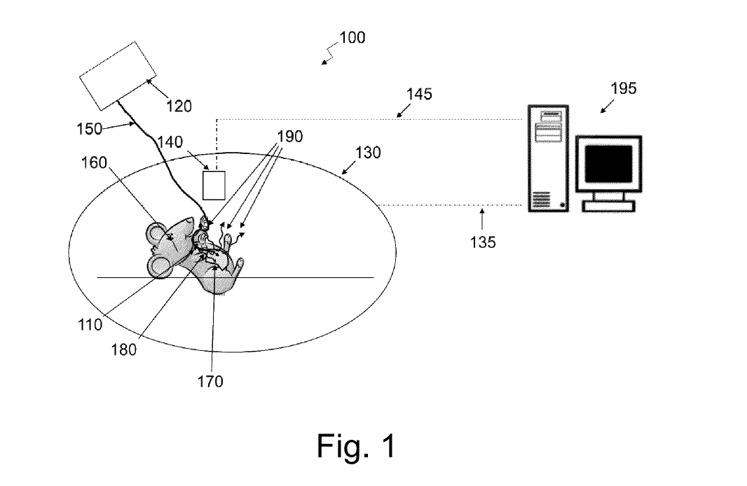

In reference to FIG. 1, which shows one embodiment (100) of the system, the subject (110), or the portion of the subject containing the volume of interest, is placed within an MRI imaging device (130). An optical fiber (150) connectable to an appropriate light source (120) is passed into the subject through incision 160 to a position in proximity with the volume of interest (170), where the provided light (180) causes fluorescence (190) of fluorescent material in the volume of interest. This fluorescent light is detected with sensors/photon detector (140) outside the body of the subject.

The optical fiber (150) can be introduced into the body through a cannula or a trocar, either an independent cannula or trocar or one forming part of a laparoscopy system, or it can lie within an incision without trocar or cannula. It can also be introduced via a body orifice, such as the nose, mouth, anus, vagina, or urethra, or via a body orifice, as given above, and through a body tissue, either via an incision or through a cannula. One example of the last would be positioning the optical fiber within the skull by passing it via the nasal passages to the ethmoid bone and through the ethmoid bone to the interior of the skull.

Both MRI imaging device (130) and photon detector (140) are in communication with the processor (195) via communication line (135) and communication line (145), wired or wirelessly. The processor (195) receives imaging data from MRI device (130) and photon detector (140), it is configured to fuse co-registered and simultaneously acquired MRI images and fluorescence images.

According to a preferred embodiment, the processor (195) utilizes fusing techniques include rendering the images using Boolean methods of correlating and combining the images. Combining binary images using Boolean logic makes it possible to select structures or objects based on multiple criteria, such as, but not limited to, masking and thresholding. The Boolean operators commonly used are OR, AND, NOT, EXCLUSIVE OR and combinations thereof.

According to another preferred embodiment, the processor (195) utilizes fuzzy logic and image local features for fusing acquired MRI and fluorescence images, or other techniques known in the field as described by Carpenter et al (Opt. let. Vol 15, pp 933-935, 2007). A perfect fusion process preserves the original functional characteristics and adds spatial characteristics to the image with no spatial distortion. The intensity-hue-saturation (IHS) algorithm and the retina-inspired model (RIM) fusion technique can preserve more spatial feature and more functional information content, respectively.

In the system of the present invention, the MRI images, which take several seconds to several minutes to acquire, provide structural information about body parts, such as organs, blood vessels, or tumors, in the volume of interest, while the fluorescence images, which may take a second or less to acquire, provide functional information about the body part or parts. For non-limiting example, MRI images show the shape and size of a tumor, while fluorescence image shows the locations of apoptopic cells and aggressively dividing cells within it

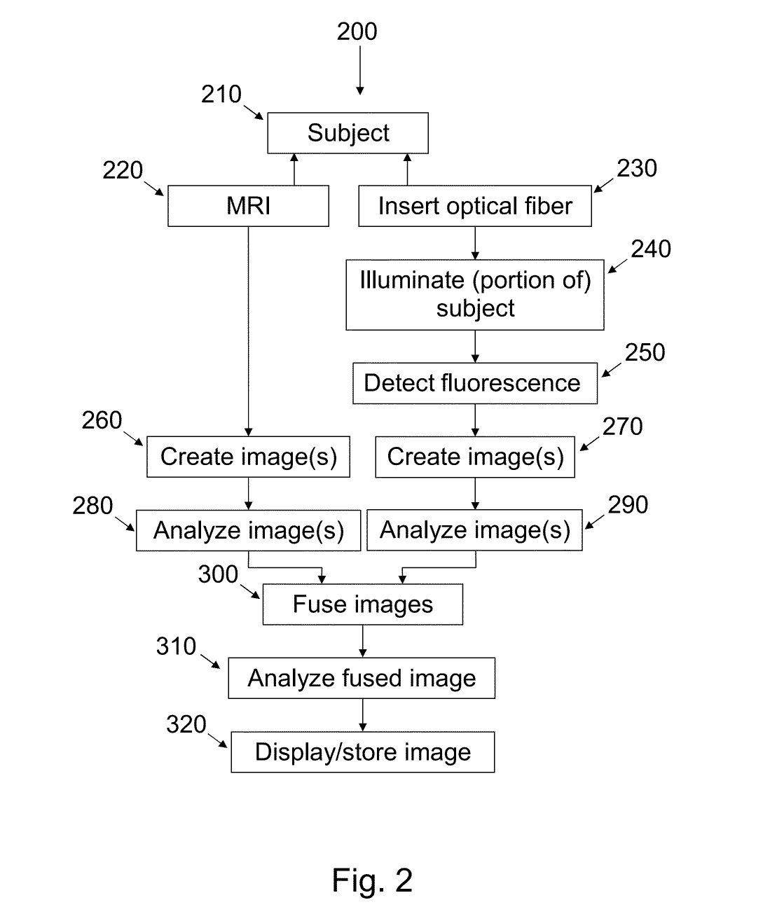

In reference to FIG. 2, a block diagram (200) of an embodiment of a method of using the system is shown. At least a volume of interest (210) within the subject is placed within the system. The volume of interest (210) can be the entire subject or a portion thereof, such as an organ or a tumor within the subject, a set of blood vessels or a set of nerves. An optical fiber is emplaced within the subject (230) in such a position that light from the fiber will activate fluorescent material within the volume of interest inside the subject. The fluorescent material can be material introduced into the subject by any of the means well known in the art, or it can be fluorescent material produced by the subject. The volume of interest is illuminated (240) via the optical fiber and the resulting fluorescence is detected by a detector outside the body of the subject (250). An image or images are created (270) of the volume of interest, using the detected fluorescence, and the image or images are analyzed (290).

An MRI scan or scans of the volume of interest in the subject is made (220). An image or images is created (260) of the volume of interest from the MRI scan, and the image or images are analyzed (280).

The MRI and fluorescence images are then fused (300), using techniques well known in the art, and the combined image is analyzed (310) and displayed or stored for later use (320).

Optical fibers are most commonly silica glass, but can also be made from fluorozirconate glass, fluoroaluminate glass, chalcogenide glass, sapphire, and polymers. The most common polymer optical fibers (POF) are (1) polymethylmethacrilate (PMMA) core with fluorinated polymer cladding, although other POF include: PMMA or Polystyrene core with silicone resin cladding, perfluorinated polymer (mainly polyperfluorobutenylvinylether) POFs, and microstructured polymer optical fibers (mPOF), which are a type of photonic crystal fiber.

1. A hybrid imaging system (100) for imaging a volume of interest of a subject, said hybrid imaging system comprises:
a. a magnetic resonance imaging (MRI) device (130) for accommodating said volume of interest of said subject and for acquiring at least one MRI image thereof;
b. a photon transmitter (150) selected from the group consisting of an optical fiber, a cannula, a light pipe, a light tube, and any combination thereof; said photon transmitter (150) having one end connected to a light source and a second end introducible into said volume of interest of said subject while said predetermined portion of said subject is placed within said MRI device, said photon transmitter being specifically adapted to being guided from within said predetermined portion of said subject;
c. at least one imaging photon detector (140) selected from the group consisting of a CCD array, a camera, a photoconductive detector array, a photovoltaic detector array, a quantum dot array, a superconducting single-photon detector array, a photovoltaic cell array, a phototube array, and any combination thereof; said photon detector (140) located functionally solely outside and substantially proximate to said predetermined portion of said animal body for acquiring at least one fluorescence image of said volume of interest of said subject substantially simultaneously with acquiring of said MRI image, by detecting fluorescence emission from an external surface of said subject, said fluorescence is excited within said subject upon illumination by light transmitted by said photon transmitter; and
d. a processor adapted to fuse said acquired MRI image and said at least one acquired fluorescence image, thereby generating a rendered MRI image of said volume of interest of said subject;
wherein said hybrid imaging system substantially simultaneously acquires said at least one magnetic resonance image and said at least one fluorescence images of said volume of interest of said subject.

2. The hybrid imaging system of claim 1, wherein said photon transmitter is an optical fiber selected from the group consisting of silica glass fiber, fluorozirconate glass fiber, fluoroaluminate glass fiber, chalcogenide glass fiber, sapphire fiber, and polymer optical fiber.

3. The hybrid imaging system of claim 1, wherein said processor utilizes Boolean logic techniques including operators selected from the group consisting of OR, AND, NOT, EXCLUSIVE OR, and any combination of said operators, for facilitating the correlation and fusion of images.

4. The hybrid imaging system of claim 1, wherein said processor utilizes fusing techniques including fuzzy logic, the intensity-hue-saturation (IHS) algorithm, the retina-inspired model (RIM) fusion technique and any combination thereof, for facilitating the correlation and fusion of images.

5. The hybrid imaging system of claim 1, wherein said photon transmitter is adapted to enter said subject through an orifice selected from the group consisting of a cannula inserted in said subject, a trocar inserted in said subject, a laparoscopy system inserted in said subject, the nose, the mouth, the anus, the vagina, the urethra, the ear, and any combination thereof.

6. The hybrid imaging system of claim 1, wherein photons transmitted by said photon transmitter are in a range selected from the group consisting of X-rays, far ultraviolet, near ultraviolet, visible light, near infrared and far infrared.

7. The hybrid imaging system of claim 1, wherein magnets in said MRI imaging device are selected from the group consisting of permanent magnets, superconducting magnets, and any combination thereof.

8. A method of imaging a volume of interest of a subject, comprising steps of:
a. providing a magnetic resonance imaging (MRI) system (130) for accommodating said volume of interest of said subject and for acquiring at least one MRI image;
b. transmitting photons utilizing means selected from the group consisting of an optical fiber, a cannula, a light pipe, a light tube, and any combination thereof; said photon transmitting means utilizes one end thereof connected to a light source and a second end thereof introducible into said volume of interest of said subject while said subject is placed within said MRI device, said photon transmitting means specifically adapted to being guided from within said portion of said animal body;
c. detecting at least one photon-induced image (140) utilizing means selected from the group consisting of a CCD array, a camera, a photoconductive detector array, a photovoltaic detector array, a quantum dot array, a superconducting single-photon detector array, a photovoltaic cell array, a phototube array, and any combination thereof; said detecting of at least one photon-induced image being functionally accomplished solely outside and substantially proximate said predetermined portion of said animal body for obtaining at least one fluorescence image of said predetermined portion of said animal body substantially simultaneously with the said obtaining of said MRI image, said detecting of fluorescence emission occurring from an external surface of said subject, said fluorescence being excited within said animal body upon illumination by light transmitted by said photon transmitter; and
d. processing and fusing both said at least one MRI image and said at least one acquired fluorescence image, thereby generating a rendered MRI image of said volume of interest of said subject;
wherein said at least one magnetic resonance image and said at least one fluorescence images of said volume of interest of said subject are acquired substantially simultaneously.

9. The method of claim 8, additionally comprising a step of selecting the optical fiber from the group consisting of silica glass fiber, fluorozirconate glass fiber, fluoroaluminate glass fiber, chalcogenide glass fiber, sapphire fiber, and polymer optical fiber.

10. The method of claim 8, additionally comprising a step of emplacing said fiber within said body through an orifice selected from the group consisting of a cannula inserted in the animal, a trocar inserted in the animal, a laparoscopy system inserted in the animal, the nose, the mouth, the anus, the vagina, the urethra, the ear, and any combination thereof.

11. The method of claim 8, additionally comprising a step of selecting the range of said photons from at least one of the group consisting of X-rays, far ultraviolet, near ultraviolet, visible light, near infrared and far infrared.

12. The method of claim 8, further comprising a step of selecting Boolean logic operators selected from the group consisting of OR, AND, NOT, EXCLUSIVE OR, and any combination of said operators, for facilitating the correlation and fusion of images.

13. The method of claim 8, further comprising a step of selecting fusing techniques including fuzzy logic, the intensity-hue-saturation (IHS) algorithm, the retina-inspired model (RIM) fusion technique and any combination thereof, for facilitating the correlation and fusion of images.

14. The method of claim 8, additionally comprising a step of selecting magnets in said MRI imaging device from the group consisting of permanent magnets, superconducting magnets, and any combination thereof.
